(19) United States
(12) Patent Application Publication
(10) Pub. No.: US 2008/0215098 A1
(43) Pub. Date: Sep. 4, 2008

(54) POROUS IMPLANT

(76) Inventors: Thomas Imwinkelried, Seltisberg (CH); Lukas Giger, Basel (CH)

Correspondence Address: SCHWEGMAN, LUNDBERG & WOESSNER / SYNTHES, P.O. BOX 2938, MINNEAPOLIS, MN, 55402, US

(21) Appl. No.: 12/027,551

(22) Filed: Feb. 7, 2008

Related U.S. Application Data
(63) Continuation of application No. PCT/CH2005/000466, filed on Aug. 10, 2005.

(51) Int. Cl. A61B 17/86; C04B 33/32

(52) U.S. Cl. 606/301; 264/671

(57) ABSTRACT

An implant comprising a shaped body having a first region with a mean porosity P₂ and a second region with a mean porosity P₃<P₂; wherein the second region with the lower mean porosity P₃ is effective for handling and fixation of the implant.

### RELATED APPLICATION

This application is a Continuation under 35 U.S.C. § 111(a) of International Application Serial No. PCT/CH2005/000466, filed Aug. 10, 2005, and published on Feb. 15, 2007 as WO 2007/016796 A1, the contents of which are incorporated herein by reference.

### FIELD

The invention relates to an implant according to the preamble of claim 1, which is an Implant with a shaped body.

Such implants may be used in particular in the field of trauma surgery, as spinal implants or as maxillo-facial implants.

### IN THE DRAWINGS

FIG. 1 shows a perspective view of an embodiment of the shaped implant according to the invention;

FIG. 2 shows a top view on the embodiment of the shaped implant shown in FIG. 1 in the green state;

FIG. 3 shows a top view on the embodiment of the shaped implant shown in FIGS. 1 and 2 in the final state after being sintered;

FIG. 4 shows a sectional view of another embodiment of the shaped implant according to the invention in the green state;

FIG. 5 shows a sectional view of the embodiment of the shaped implant shown in FIG. 4 in the final state together with a fixations screw; and

FIG. 6 shows a frontal view of the inlay according to the embodiment shown in FIGS. 4 and 5.

### DETAILED DESCRIPTION

In order to handle such implants and to anchor them to bone, a countersunk threaded bore in the sintered body of the implant is applied. However, due to the high surface roughness of that body, the manipulation with an instrument and the introduction of fixation means, like fixation screws may lead to the abrasion of particles from the implant.

The aim of the present invention is to provide a means for a stable mechanical attachment to a porous implant and to avoid the above described production of abrasion particles during handling and/or fixation of the implant.

The invention solves the posed problem with an implant that displays the features of claim 1, which is as follows: Implant (1) with a shaped body characterized in that A) said body has a first region (2) with a mean porosity P₂ and a second region (3) with a mean porosity P₃<P₂; and that B) said second region (3) with the lower mean porosity P₃ is designed for handling or fixation of the implant (1).

Thanks to the second region of the implant which has a lower mean porosity than the first region of said implant abrasion of particles of sintered material during handling or fixation of the implant can be avoided.

In metallurgical and ceramic technology, numerous methods for producing shaped bodies with interconnecting pores are known. Typical methods of manufacture of shaped sintered bodies are disclosed
Titanium foam: e.g. in DE-A 196 38 927, WO 03/101647 A2 and WO 01/19556 whose content is incorporated in this application.
Porous nitinol: U.S. Pat. No. 5,986,169
Porous tantalum: U.S. Pat. No. 5,282,861, EP 0560 279
Porous metals and metal coatings for implants WO 02/066693

In order to achieve a suitable surface structure for fixation e.g. by means of a bone screw or for manipulation of the implant by means of an instrument an inlay made of a fully dense material e.g. a titanium inlay may be embedded in a corresponding aperture in the implant. The titanium inlay may be provided with means, e.g. a cavity that allows cooperation with a tool for handling said implant or reception of fixation means for fixation of said implant, whereby these means permit high geometrical tolerances for secure engagement of a tool or fixation means and do not lead to abrasion of titanium particles during manipulation or fixation. Before the sintering process is effected, the inlay and the “green” state titanium foam are combined. Thereto, the inlay may be inserted in a bore hole in the green body, whereby the inlay may have a clearance in the bore hole or may be loosely attached to the green body. Due to the shrinkage of the titanium foam during the sintering process the inlay is strongly clamped in the post sintered state of the implant.

The inlay may be kept in its position in the bore hole during the sintering process by means of the gravitational force in case of being inserted in a bore hole with a clearance or by means of a loose seat of small projections at the outer surface of the inlay that contact the wall of the bore hole in the green body.

Alternatively, with a tough and ductile material like titanium, the pore walls of the foam structure of the first region of the implant may be “smeared” during traditional machining (e.g. turning, milling, etc.). The smearing effect is being used to get smoother surfaces at the fixation interface, e.g. the means allowing cooperation with a tool for handling said implant or reception of fixation means for fixation of said implant. Said means are preferably being configured as interior thread. However, an implant with a porous structure, which has been machined after the sintering process, is very difficult to clean. The contamination and the smearing effect due to the machining can be avoided by alternative processes such as wire EDM (electro-discharge machining) or water-jet cutting. Both processes allow to keep an open-porous structure at the surface.

In a preferred embodiment the first region of the body comprises the same material as the second region. By means of the gradient of the porosity in the body the second region of the body is manufacturable such that abrasion of particles during handling or fixation of the implant can be avoided.

In another embodiment the first region of the body comprises a different material compared to the second region. Therewith the advantage is achievable that a material with a lower porosity may be selected for the second region of the body allowing a handling or fixation of the implant without abrasion of particles.

In a further embodiment at least one of the mean porosities P₃<P₂ has a gradient.

In yet another embodiment the mean porosity P₂ of the first region of the body is in the range of 30-90%, preferably of 50-70%. The advantage of a mean porosity in said range is an optimal combination of mechanical properties and maximum possible porosity for the bone ingrowth.

Preferably, the mean porosity P₃ of the second region of the body is below 10%, preferably below 2%. The advantage is that this porosity allows to obtain optimally smooth surfaces which do not produce any abrasive particles.

In yet a further embodiment the second region is in the form of an inlay which may be combined with the first region before the sintering process. After the sintering process the inlay is strongly clamped by the sintered first region due to their shrinkage.

In another embodiment the second region is provided with means allowing cooperation with a tool for handling said implant or reception of fixation means for fixation of said implant.

In a further embodiment the first region of the body comprises an inorganic material, preferably a metallic or ceramic material. Said inorganic material may be chosen from the groups of biocompatible metals or sintered ceramics, preferably biocompatible steel, titanium and titanium alloys, tantalum and tantalum alloys, biocompatible NiTi-alloys, magnesium and magnesium alloys.

In yet another embodiment the first region comprises an open-porous metallic foam with interconnected porosity. Preferably, said metallic foam is produced by a powder metallurgical process or by a coating process or by combustion synthesis or by other known foam production processes.

In yet a further embodiment the first region of the body comprises a material obtained by powder metallurgy using the space holder technique to produce green compact and a subsequent porous sintered body.

In another embodiment the second region of the body comprises a biocompatible metal or metal alloy, preferably Ti, steel, Ta, biocompatible NiTi-alloys.

In a further embodiment the second region of the body has a minor surface roughness compared to the first region.

In yet another embodiment the second region of the body has a higher density compared to the first region.

A first method for manufacture of an implant according to the invention includes the step that an inlay comprising a material with said mean porosity P₃ is placed into an opening of a green compact comprising a material with said mean porosity P₂ before sintering of said net-shape implant, whereby said implant is net-shape.

In a preferred embodiment of the method the inlay is loosely placed into an opening of said green compact and wherein said inlay is standing on a surface of said green body.

In another embodiment of the method inlay is placed inside said opening of the green compact touching several walls of the compact and where the inlay is mainly withhold by friction.

A second method for manufacture of an implant according to the invention includes the step that an inlay comprising a material with said mean porosity P₃ is placed inside an aperture of said first region of said implant after sintering of said first region by force or using thermal expansion differences.

The invention and additional configurations of the invention are explained in even more detail with reference to the partially schematic illustration of several embodiments.

The following examples will further explain the implant according to the invention and its manufacture.

### EXAMPLE 1

### Implant with an Inlay Obtained by Net-Shape Sintering

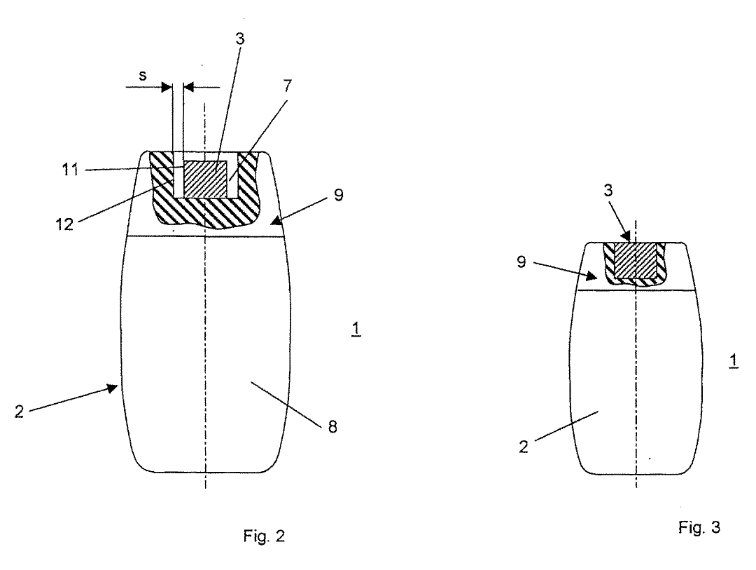

A first region 2 of the implant in the form of a “green” state titanium foam 8 and a second region 3 of the implant made of a fully dense material in the form of a titanium inlay are combined before the sintering process (FIG. 2). As shown in FIG. 2 the second region 3 in form of an inlay is loosely placed in a countersunk bore 7 of the “green” state titanium foam 8.

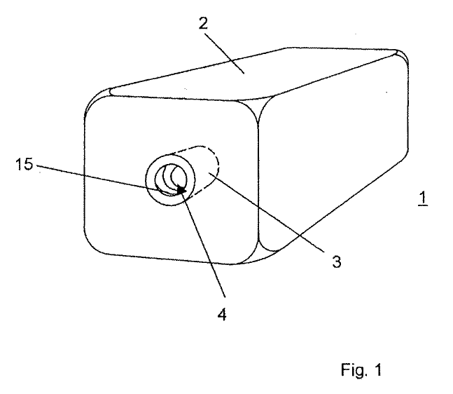

The second region 3, i.e. the inlay comprises means 4 (FIG. 1) allowing cooperation with a tool for handling the implant or for receiving a fixation means for fixation of the implant 1 e.g. at a bone. In order to avoid a production of abrasion particles during manipulation and/or fixation of the implant 1 the material of the second region, i.e. of the inlay has a lower mean porosity P₃ (e.g. below 10%) compared to the surrounding green body (e.g. between 30 and 90%). The attachment of the second region 3 in form of an inlay to the first region 2 in a mechanically stable manner is achieved by means of sintering the first region 2 together with the combined second region 3, i.e. the inlay. Due to the shrinkage of the first region 2 in the form of a “green” state titanium foam 8 during the sintering process, the second region 3, i.e. the inlay is strongly clamped by the sintered first region 2 (FIG. 3).

### EXAMPLE 2

### Implant with an Inlay Obtained by Post-Sintering Treatment

Alternatively, the second region 3, in form of a fully dense fixation inlay is inserted into the foam structure of the sintered first region 2 by force (mechanically) or by shrinking the first region 2 onto the second region 3, i.e. the inlay. After sintering the first region 2, the second region 3, i.e. the inlay is inserted into a countersunk bore 7 (FIG. 2) in the sintered first region 2 either mechanically with a press-fit or using differences in thermal expansion between the two regions 2,3 (i.e. to heat the outer first region 2 and/or to shrink the second region 3, i.e. the inlay by cooling). In order to avoid the abrasion of particles the material of the second region 3 preferably has a porosity below 10% while the material of the surrounding first region 2 preferably has a porosity between 30% and 90%.

### EXAMPLE 3

### Implant with an Inlay Held in Place by Gravity During the Green State

FIGS. 1 to 3 show a hollow second region 3, i.e. an inlay being provided with an interior thread 15 (FIG. 1) and made of titanium alloy (TAN) within the reinforced layer 9 of a first region 2 in the form of a titanium foam, whereby the reinforced layer 9 has a porosity of 10-20%. The purpose of the second region 3, i.e. the inlay is to serve as an interface with the implant holder (not shown) which is screwed into the interior thread 15 in the implant 1.

Before sintering, the threaded second region 3, i.e. the inlay is placed manually into the countersunk bore 7 of the upright standing first region 2 in the form of a “green” state titanium foam 8 (FIG. 2) In case of the embodiment according to FIGS. 2 and 3 there is a clearance “s” between to outer wall 11 of the second region 3, i.e. the inlay and the wall 12 of the countersunk bore 7. During the sintering process the second region 3, i.e. the inlay is kept in its position by means of the gravitational force. During sintering, the reinforced layer 9 (porosity of 10-20%) shrinks by about 10% and bonds to the second region 3, i.e. the inlay (porosity below 10%).

### EXAMPLE 4

### Implant with an Inlay Held in Place by Friction During the Green State

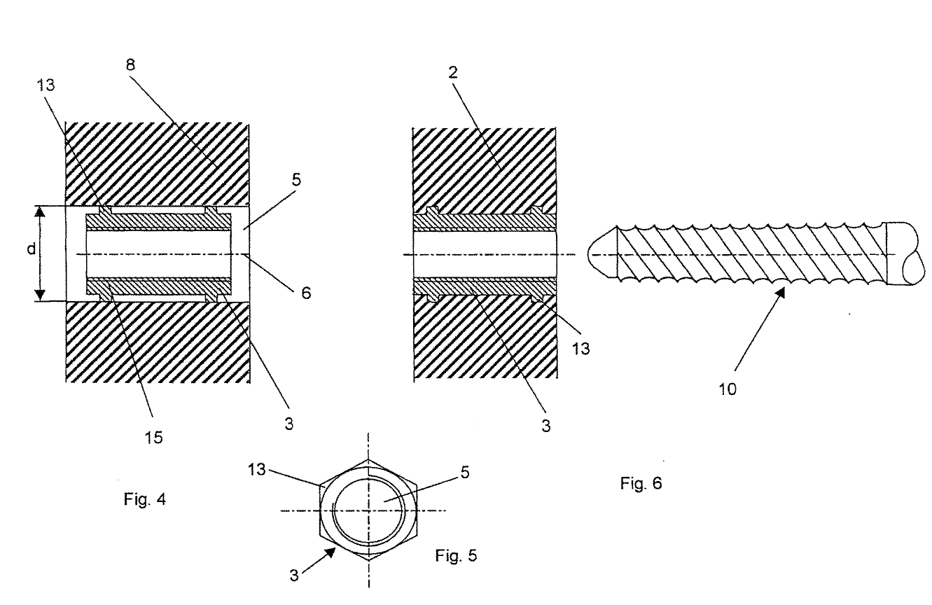

In case of the embodiment according to FIGS. 4 to 6 the outer wall 11 of the second region 3, i.e. the inlay is provided with small protrusions 13 in the form of two hexagonal rings being arranged concentrically to the central axis 6 of the second region 3, i.e. the inlay. The diameter d of the cavity 5 is slightly smaller or equal to the width across the edges 14 of the hexagonal rings such that the second region 3, i.e. the inlay is loosely attached to the “green” state titanium foam 8 before the sintering process. Furthermore, the hexagonal rings allow an axial and rotational positive fit between the second region 3, i.e. the inlay and the first region 2 after the sintering process. A bone screw 10 is screwable into the interior thread 15 in the cavity 5 in the second region 3, i.e. the inlay. By means of the bone screw 10 the implant 1 is apt to be rigidly fixed in a bone during the surgical procedure.

The threaded second region 3, i.e. the inlay is made of commercially pure titanium with a porosity of preferably below 10%. During sintering, the “green” state titanium foam 8 (FIG. 4) with a porosity of about 60% shrinks by about 15% in both directions and ends up embracing the second region 3, i.e. the inlay in a solid link.

Embodiments also include Implant embodiments (1) with a shaped body

characterized in that A) said body has a first region (2) with a mean porosity P₂ and a second region (3) with a mean porosity P₃<P₂; and that B) said second region (3) with the lower mean porosity P₃ is designed for handling or fixation of the implant (1).

For some implant embodiments, the said first region (2) comprises the same material as said second region (3).

For some embodiments, the said first region (2) comprises a different material compared to said second region (3).

For some embodiments the said at least one of said mean porosities P₃<P₂ has a gradient.

For some embodiments, the implant (1) is characterized in that the mean porosity P₂ is in the range of 30-90%, preferably of 50-70%.

For some embodiments the implant (1) is characterized in that the mean porosity P₃ is below 10%. For some embodiments the mean porosity is below 2

For some embodiments, the implant (1) is characterized in that said second region (3) is in the form of an inlay.

For some embodiments the implant (1) is characterized in that said second region (3) is provided with means (4) allowing cooperation with a tool for handling said implant (1) or reception of fixation means for fixation of said implant (1).

For some embodiments, the implant (1) is characterized in that said first region (2) comprises an inorganic material, preferably a metallic or ceramic material.

For some embodiments, the implant (1) is characterized in that the inorganic material is chosen from the groups of biocompatible metals or sintered ceramics, preferably biocompatible steel, titanium and titanium alloys, tantalum and tantalum alloys, biocompatible NiTi-alloys, magnesium and magnesium alloys.

For some embodiments, the implant (1) is characterized in that the first region (2) comprises an open-porous metallic foam with interconnected porosity.

For some embodiments, the implant (1) is characterized in that the metallic foam is produced by a powder metallurgical process or by a coating process or by combustion synthesis or by other known foam production processes.

For some embodiments, the implant (1) is characterized in that the first region (2) comprises a material obtained by powder metallurgy using the space holder technique to produce green compact and a subsequent porous sintered body.

For some embodiments, the implant (1) is characterized in that the second region (3) comprises a biocompatible metal or metal alloy, preferably Ti, steel, Ta, biocompatible NiTi-alloys.

For some embodiments the implant (1) is characterized in that the second region (3) has a minor surface roughness compared to said first region (2).

For some embodiments, the implant (1) is characterized in that the second region (3) has a higher density compared to said first region (2).

Some method embodiments are characterized in that an inlay comprising a material with said mean porosity P₃ is placed into an opening of a green compact comprising a material with said mean porosity P₂ before sintering of said net-shape implant, whereby said implant is net-shape.

Some method embodiments are characterized in that the inlay is loosely placed into an opening of said green compact and wherein said inlay is standing on a surface of said green body.

Some method embodiments are characterized in that said inlay is placed inside said opening of the green compact touching several walls of the compact and where the inlay is mainly withhold by friction.

Some method for manufacture of an implant are characterized in that an inlay comprising a material with said mean porosity P₃ is placed inside an aperture of said first region (2) of said implant after sintering of said first region (2) by force or using thermal expansion differences.

1. An implant comprising a shaped body having a first region with a mean porosity P₂ and a second region with a mean porosity P₃<P₂; wherein the second region with the lower mean porosity P₃ is effective for handling and fixation of the implant.

2. The Implant of claim 1, wherein the first region comprises the same material as said second region.

3. The implant of claim 1, wherein the first region comprises a different material compared to said second region.

4. The implant of claim 1, wherein the at least one of the mean porosities P₃<P₂ has a gradient.

5. The implant of claim 1, wherein the mean porosity P₂ is in a range of 30-90%.

6. The implant of claim 1, wherein the mean porosity P₃ is below 10%.

7. The implant of claim 1, wherein the second region is in the form of an inlay.

8. The implant of claim 1, wherein the second region comprises means for allowing cooperation with a tool for handling the implant or reception of fixation means for fixation of the implant.

9. The implant of claim 1 wherein the first region comprises an inorganic material.

10. The implant of claim 9, wherein the inorganic material is selected from the groups of biocompatible metals or sintered ceramics.

11. The implant of claim 1 wherein the first region comprises an open-porous metallic foam with interconnected porosity.

12. The implant of claim 11, wherein the metallic foam is made by a powder metallurgical process or by a coating process or by combustion synthesis or by other known foam production processes.

13. The implant of claim 1, wherein the first region comprises a material obtained by powder metallurgy using a space holder technique to produce green compact and a subsequent porous sintered body.

14. The implant of claim 1, wherein the second region comprises a biocompatible metal or metal alloy.

15. The implant of claim 1, wherein the second region has a minor surface roughness compared to said first region.

16. The implant of claim 1, wherein the second region has a higher density compared to said first region.

17. A method for manufacture of an implant comprising a shaped body having a first region with a mean porosity P₂ and a second region with a mean porosity P₃<P₂; wherein the second region with the lower mean porosity P₃ is effective for handling and fixation of the implant, characterized in that an inlay comprising a material with said mean porosity P₃ is placed into an opening of a green compact comprising a material with said mean porosity P₂ before sintering of said net-shape implant, the implant having a net-shape.

18. The method of claim 17, wherein the inlay is loosely placed into an opening of said green compact and wherein said inlay is standing on a surface of said green body.

19. The method of claim 17, wherein the inlay is placed inside the opening of the green compact touching several walls of the compact and where the inlay is mainly withhold by friction.

20. The method for manufacture of an implant comprising a shaped body having a first region with a mean porosity P₂ and a second region with a mean porosity P₃<P₂; wherein the second region with the lower mean porosity P₃ is effective for handling and fixation of the implant, characterized in that an inlay comprising a material with said mean porosity P₃ is placed inside an aperture of said first region (2) of said implant after sintering of said first region (2) by force or using thermal expansion differences.
